(19) United States
(12) Patent Application Publication
(10) Pub. No.: US 2007/0178450 A1
(43) Pub. Date: Aug. 2, 2007

(54) Method and apparatus for determining level of microorganisms using bacteriophage

(75) Inventors: John H. Wheeler, Boulder, CO (US); Jon C. Rees, Longmont, CO (US); Gregory S. Gaisford, Denver, CO (US)

Correspondence Address: PATTON BOGGS LLP, 1801 CALFORNIA STREET, SUITE 4900, DENVER, CO, 80202, US

(73) Assignee: MicroPhage (TM) Incorporation, Longmont, CO (US)

(21) Appl. No.: 11/698,673

(22) Filed: Jan. 25, 2007

Related U.S. Application Data
(60) Provisional application No. 60/762,749, filed on Jan. 27, 2006.
(60) Provisional application No. 60/794,652, filed on Apr. 24, 2006.
(60) Provisional application No. 60/800,922, filed on May 15, 2006.

(51) Int. Cl. C12Q 1/70; C12Q 1/68

(52) U.S. Cl. 435/5; 435/6

(57) ABSTRACT

A predetermined amount of parent bacteriophage capable of infecting a target microorganism is added to a sample to create a bacteriophage-exposed sample; the sample is incubated for a defined incubation time and assayed to determine the level of a bacteriophage or bacterial marker in the sample; and if the measured marker level has increased, then the initial concentration of the microorganism exceeds a specific threshold value. An antibiotic in different concentrations is added to different and separate portions of the sample and tested to determine if the bacteriophage marker is present and thereby determine the Minimum Inhibitory Concentration (MIC) of a given antibiotic. The antibiotic preferably is an antibiotic that inhibits DNA replication or protein synthesis.

### CROSS-REFERENCE TO RELATED APPLICATIONS

This Application is a Non-Provisional of Provisional (35 USC 119(e)) Application No. 60/762749 filed on Jan. 27, 2006. This Application also is a Non-Provisional of Provisional (35 USC 119(e)) Application No. 60/794652 filed on Apr. 24, 2006. This Application also is a Non-Provisional of Provisional (35 USC 119(e)) Application No. 60/800922 filed on May 15, 2006.

### FIELD OF THE INVENTION

The invention relates generally to the field of quantifying microscopic living organisms, and more particularly to the quantifying of microorganisms using bacteriophage and determining the antibiotic susceptibility of those microorganisms.

### BACKGROUND OF THE INVENTION

Classical microbiological methods are still the most commonly used techniques for identifying and quantifying specific bacterial pathogens. These methods are generally easy to perform, do not require expensive supplies or laboratory facilities, and offer high levels of selectivity; however, they are slow. Classical microbiological methods are hindered by the requirement to first grow or cultivate pure cultures of the targeted organism, which can take many hours to days. This time constraint severely limits the ability to provide a rapid and ideal response to the presence of virulent strains of microorganisms. The extensive time it takes to identify microorganisms using standard methods is a serious problem resulting in significant human morbidity and increased economic costs. Thus, it is not surprising that much scientific research has been done and is being done to overcome this problem.

Bacteriophage amplification has been suggested as a method to accelerate microorganism identification. See, for example, U.S. Pat. No. 5,985,596 issued Nov. 16, 1999 and U.S. Pat. No. 6,461,833 B1 issued Oct. 8, 2002, both to Stuart Mark Wilson; U.S. Pat. No. 4,861,709 issued Aug. 29, 1989 to Ulitzur et al.; U.S. Pat. No. 5,824,468 issued Oct. 20, 1998 to Scherer et al.; U.S. Pat. No. 5,656,424 issued Aug. 12, 1997 to Jurgensen et al.; U.S. Pat. No. 6,300,061 B1 issued Oct. 9, 2001 to Jacobs, Jr. et al.; U.S. Pat. No. 6,555,312 B1 issued Apr. 29, 2003 to Hiroshi Nakayama; U.S. Pat. No. 6,544,729 B2 issued Apr. 8, 2003 to Sayler et al.; U.S. Pat. No. 5,888,725 issued Mar. 30, 1999 to Michael F. Sanders; U.S. Pat. No. 6,436,661 B1 issued Aug. 20, 2002 to Adams et al.; U.S. Pat. No. 5,498,525 issued Mar. 12, 1996 to Rees et al.; Angelo J. Madonna, Sheila VanCuyk and Kent J. Voorhees, “Detection Of Esherichia Coli Using Immunomagnetic Separation And Bacteriophage Amplification Coupled With Matrix-Assisted Laser Desorption/Ionization Time-Of-Flight Mass Spectrometry”, Wiley InterScience, DOI:10.1002/rem.900, 24 Dec. 2002; and United States Patent Application Publication No. 2004/0224359 published Nov. 11, 2004. Bacteriophage are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called lytic bacteriophage, rupture the host bacterium, thereby releasing the progeny phage into the surrounding environment to seek out other bacteria. The total time for infection of a bacterium by parent phage, phage multiplication (amplification) in the bacterium to produce progeny phage, and release of the progeny phage after lysis can take as little as an hour depending on the phage, the bacterium, and the environmental conditions. Thus, it has been proposed that the use of bacteriophage amplification in combination with a test for bacteriophage or a bacteriophage marker may be able to significantly shorten the assay time as compared to a traditional substrate-based identification.

A simple identification of the presence of a microorganism may be insufficient to determine if a problem exists, because, in the case of many microorganisms, their presence at a low concentration is often expected, and is not necessarily an indication of an unhealthy or unsafe sample. However, in conventional practice, determination of the quantity of a microorganism that is present is significantly slower than identification. This results in much economic loss because, to be safe, procedures such as medical treatment or destruction of food are begun before the quantity of microorganisms that are present are determined, which procedures are often unnecessary and, therefore, inefficient and wasteful. Thus, there remains a need for a faster method of determining the concentration of microorganisms that are present in a sample.

### BRIEF SUMMARY OF THE INVENTION

The invention solves the above problems, as well as other problems of the prior art, by using bacteriophage to provide a quantitative determination of the amount of the microorganism that is present in a sample. The inventors have discovered that if a prescribed amount of parent bacteriophage specific to a target microorganism is added to a sample that includes the target microorganism, the time it takes to develop an amplified level of bacteriophage or bacterial marker can be correlated with the initial quantity of target microorganism in the sample. Preferably, the certain level of marker is the minimum detectable level of the marker.

The invention maybe used to quickly determine whether the concentration of the target microorganism is above or below a threshold level as, for example, a level above which health problems can occur. For a given amount of parent bacteriophage added to a sample, the time it takes to develop a characteristic amplified bacteriophage or bacterial marker level depends on the initial bacterial concentration in the sample. Thus, to determine if the bacterial concentration in an unknown sample is above or below a threshold concentration, parent bacteriophage at a known concentration is added to the sample and the bacteriophage or bacterial marker is assayed at a defined time later. If an increase marker level is detected, the initial bacterial concentration in the sample exceeds the threshold concentration. If not, then the concentration is below the threshold concentration.

The invention provides a method of determining if a threshold concentration of a target microorganism is present in a sample to be tested, the method comprising: (a) combining with the sample a predetermined amount of parent bacteriophage capable of infecting the target microorganism to create a bacteriophage exposed sample; (b) providing incubation conditions to the bacteriophage-exposed sample sufficient to allow the parent bacteriophage to infect the target microorganism; (c) waiting a predetermined time period such that, if the target microorganism is present in the sample at or above a threshold concentration, an amplified bacteriophage marker will be detectable in the sample; and (d) assaying the exposed sample to determine if the bacteriophage marker is amplified. Preferably, the target microorganism is bacteria. Preferably, the bacteriophage marker comprises an element selected from the group consisting of the bacteriophage, bacteriophage nucleic acid, bacteriophage protein, and a portion of a bacteriophage nucleic acid or a bacteriophage protein. Preferably, the parent bacteriophage has been genetically modified to add the marker. Preferably, the parent bacteriophage is added in an amount below the detection limit of the bacteriophage marker.

The invention also provides a method of determining if a threshold concentration of a target microorganism is present in a sample to be tested, the method comprising: (a) combining with the sample a predetermined amount of parent bacteriophage capable of infecting the target microorganism to create a bacteriophage-exposed sample; (b) providing incubation conditions to the bacteriophage-exposed sample sufficient to allow the parent bacteriophage to infect the target microorganism; (c) waiting a predetermined time period such that, if the target microorganism is present in the sample at or above a threshold concentration, a bacterial marker will be detectable in the sample; and (d) assaying the exposed sample to determine if the bacterial marker is detectable. Preferably, the target microorganism is a bacterium. Preferably, the bacterial marker comprises an element selected from the group consisting of: cell wall debris, bacterial nucleic acids, proteins, small molecules, or enzymes that are released when a phage lyses the bacteria.

The invention also provides a method of determining the initial quantity of a microorganism present in a sample, the method comprising: (a) combining with the sample a predetermined amount of parent bacteriophage capable of infecting the target microorganism to create a bacteriophage exposed sample; (b) providing incubation conditions to the bacteriophage-exposed sample sufficient to allow the parent bacteriophage to infect the target microorganism and create an amplified bacteriophage marker in the bacteriophage exposed sample; (c) assaying the bacteriophage marker in the exposed sample to determine a marker level in the sample; (d) measuring a reaction time associated with the marker level; and (e) determining the initial quantity of the microorganism present in the sample using the measured reaction time. Preferably, the initial quantity comprises the concentration of the microorganism in the sample at the time of adding the parent bacteriophage. Preferably, the target microorganism is a bacterium. Preferably, the parent bacteriophage is added in an amount below the defined detection limit of the bacteriophage marker. Preferably, the determining comprises: providing a table correlating the reaction time to the initial quantity; and selecting the initial quantity from the table. Preferably, the table also correlates the predetermined amount of parent bacteriophage to the initial quantity. Preferably, the measuring comprises waiting a predetermined time; the assaying comprises establishing if the sample contains a detectable amount of the bacteriophage marker, and the determining comprises ascertaining that the initial quantity is below a threshold value. Preferably, the bacteriophage marker comprises an element selected from the group consisting of: the bacteriophage, bacteriophage nucleic acid, bacteriophage protein, and a portion of a bacteriophage nucleic acid or a bacteriophage protein. Preferably, the parent bacteriophage has been genetically modified to add the marker.

In another aspect, the invention provides a method of determining the susceptibility or resistance of a target microorganism in a sample to an antibiotic, the method comprising: (a) combining the sample with the antibiotic to create an antibiotic-exposed sample; (b) combining with the antibiotic-exposed sample a predetermined amount of parent bacteriophage capable of infecting the target microorganism to create a bacteriophage-exposed sample; (c) providing incubation conditions to the bacteriophage-exposed sample sufficient to allow the parent bacteriophage to infect the target microorganism; (d) waiting a predetermined time period such that, if the target microorganism is not susceptible or is resistant to the antibiotic, an amplified bacteriophage marker will be detected in the sample; and (e) assaying the exposed sample to determine the presence of the amplified bacteriophage marker as an indication of the susceptibility or resistance of the microorganism to the antibiotic. Preferably, the parent bacteriophage is combined in an amount below the detection limit of the bacteriophage marker. Preferably, said combining comprises diluting the concentration of said target microorganism to a level at which said bacteriophage infection will not occur immediately.

In yet another aspect, the invention provides a method of determining the susceptibility or resistance of a target microorganism in a sample to an antibiotic, the method comprising: (a) combining the sample with the antibiotic to create an antibiotic-exposed sample; (b) combining the antibiotic-exposed sample and a predetermined amount of parent bacteriophage capable of infecting the target microorganism to create a bacteriophage-exposed sample; (c) providing incubation conditions to the bacteriophage-exposed sample sufficient to allow the parent bacteriophage to infect the target microorganism and create an amplified bacteriophage marker in the bacteriophage-exposed sample; (d) assaying the bacteriophage marker in the exposed sample to determine a marker level in the sample; (e) measuring a reaction time associated with the marker level; and (f) determining the susceptibility or resistance of the target microorganism to the antibiotic using the measured reaction time.

Preferably, for the methods taught herein for determining the susceptibility or resistance of a target microorganism to an antibiotic, the antibiotic inhibits nucleic acid replication. Preferably, the antibiotic is selected from the group consisting of: flouroquinilones, such as levofloxacin and ciprofloxacin, and rifampin. Alternatively, the antibiotic inhibits protein synthesis. Preferably, the antibiotic is selected from the group consisting of: macrolides, aminoglycosides, tetracyclines, streptogramins, everninomycins, oxazolidinones, and lincosamides. Preferably, the antibiotic is added to a plurality of different and separate portions of the sample in different antibiotic concentrations. Preferably, the adding comprises adding a plurality of different antibiotics to the sample, with each of the different antibiotics added to a different and separate sample portion.

Preferably, for all the methods taught herein, the assaying comprises a colorimetric test. Preferably, the assaying comprises one or more tests selected from the group consisting of: immunoassay methods, nucleic acid amplification-based assays, DNA probe assays, aptamer-based assays, mass spectrometry, including MALDI, and flow cytometry. Preferably, the immunoassay methods are selected from the group consisting of ELISA, radioimmunoassay, immunoflouresence, lateral flow immunochromatography (LFI), flow-through assay, and a test using a SILAS surface.

The invention not only permits a rapid measurement of the quantity of a microorganism that is present in a sample, but also permits the antibiotic susceptibility or resistance of the microorganism to be rapidly determined. Numerous other features, objects, and advantages of the invention will become apparent from the following description when read in conjunction with the accompanying drawings.

### BRIEF DESCRIPTION OF THE DRAWINGS

FIG. 1a is a graph of bacteriophage concentration versus time in a sample that has an initial bacteria concentration of 10⁴ bacteria per milliliter illustrating how bacteriophage amplification can be used to determine the quantity of a microorganism as well as identify a microorganism;

FIG. 1b is a graph of bacterial debris concentration versus time in the same sample illustrated in FIG. 1a;

FIG. 2a is a graph of bacteriophage concentration versus time in a sample that has an initial bacteria concentration of 10⁶ bacteria per milliliter, but is otherwise identical to the sample of FIG. 1a;

FIG. 2b is a graph of bacterial debris concentration versus time in the same sample illustrated in FIG. 2a;

FIG. 3 is a flow chart illustrating a preferred embodiment of the method according to the invention;

FIG. 4 is a flow chart illustrating another preferred embodiment of the method according to the invention;

FIG. 5 is a graph of bacteriophage concentration versus time that illustrates how bacteriophage amplification can be used to rapidly determine antibiotic susceptibility or resistance of a microorganism;

FIG. 6 is a graph showing how long it takes for a bacteriophage marker to exceed a threshold level with different bacterial strains as a function of antibiotic concentration;

FIG. 7 is an illustration of a bacteriophage;

FIG. 8 illustrates a typical phage reproduction process as a function of time; and

FIG. 9 shows a side plan view of a lateral flow microorganism detection device according to the invention.

### DETAILED DESCRIPTION OF THE INVENTION

In this disclosure, the terms “bacteriophage” and “phage” include bacteriophage, phage, mycobacteriophage (such as for TB and para TB), mycophage (such as for fungi), mycoplasma phage or mycoplasmal phage, and any other term that refers to a virus that can invade living bacteria, fungi, mycoplasmas, protozoa, yeasts, and other microscopic living organisms and uses them to replicate itself. Here, “microscopic” means that the largest dimension is one millimeter or less. Bacteriophage are viruses that have evolved in nature to use bacteria as a means of replicating themselves. A phage does this by attaching itself to a bacterium and injecting its DNA (or RNA) into that bacterium, and inducing it to replicate the phage hundreds or even thousands of times. A particular bacteriophage will usually infect only a particular bacterium. That is, the bacteriophage is specific to the bacteria. Thus, if a particular bacteriophage that is specific to particular bacteria is introduced into a sample, and later the bacteriophage has been found to have multiplied, the bacteria to which the bacteriophage is specific must have been present in the sample. In this way, as is known in the art, bacteriophage amplification can be used to identify bacteria present in a sample.

Whether the bacteriophage has infected the bacteria is determined by an assay that can identify the presence of a bacteriophage or bacterial marker. In this disclosure, a bacteriophage marker is any biological or organic element that can be associated with the presence of a bacteriophage. Without limitation, this maybe the bacteriophage itself, a lipid incorporated into the phage structure, a protein associated with the bacteriophage, RNA or DNA associated with the bacteriophage, or any portion of any of the foregoing. In this disclosure, a bacterial marker is any biological or organic element that is released when a bacterium is lysed by a bacteriophage, including cell wall components, bacterial nucleic acids, proteins, enzymes, small molecules, or any portion of the foregoing. Preferably, the assay not only can identify the bacteriophage marker, but also the quantity or concentration of the bacteriophage or bacterial marker. In this disclosure, determining the quantity of a microorganism is equivalent to determining the concentration of the microorganism, since if you have one, you have the other, since the volume of the sample is nearly always known, and, if not known, can be determined. Determining the quantity or concentration of something can mean determining the number, the number per unit volume, determining a range wherein the number or number per unit volume lies, or determining that the number or concentration is below or above a certain critical threshold. Generally, in this art, the amount of microorganism is given as a factor of ten, for example, 2.3×10⁷ bacteria per milliliter (ml).

Some bacteriophage, called lytic bacteriophage, rupture the host bacterium, releasing the progeny phage into the environment to seek out other bacteria. The total reaction time for phage infection of a bacterium, phage multiplication, or amplification in the bacterium, through lysing of the bacterium takes anywhere from tens of minutes to hours, depending on the phage and bacterium in question and the environmental conditions. Once the bacterium is lysed, progeny phage are released into the environment along with all the contents of the bacteria. The progeny phage will infect other bacteria that are present, and repeat the cycle to create more phage and more bacterial debris. In this manner, the number of phage will increase exponentially until there are essentially no more bacteria to infect.

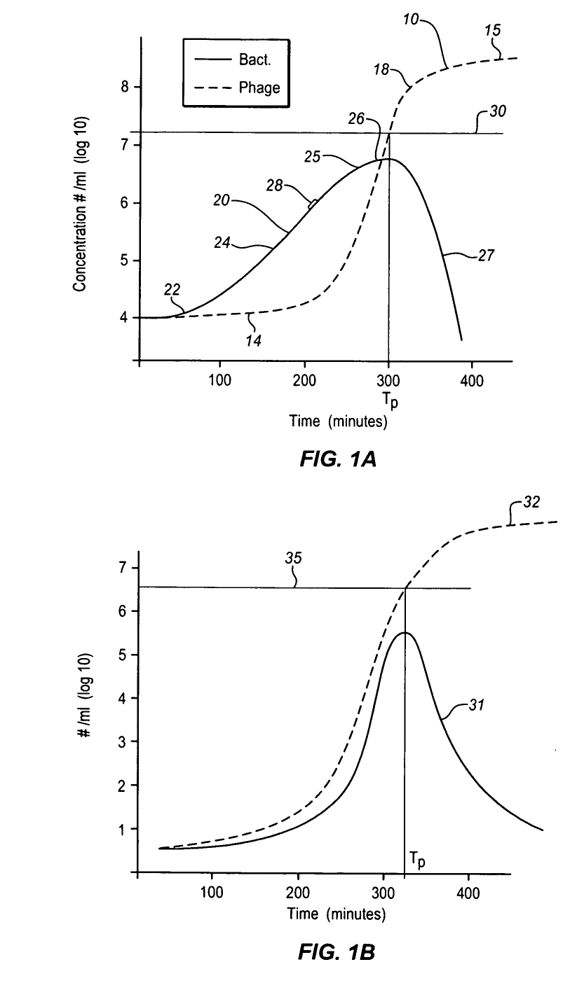

FIG. 1a includes a logarithmic graph 10 of phage concentration versus time for a test sample initially containing 10⁴ target bacteria for which the phage were specific. The figure also includes a graph 20 showing the concentration of the target bacteria versus time for the same test sample. At time zero, approximately 10⁴ lytic phage were added to the sample. The sample was then incubated. At first, the phage do not even appreciably amplify, since the probability that the phage and bacteria interact is very small at these starting concentrations. Essentially, the infection process cannot occur until there are enough bacteria present in the sample for the phage to find them. Thus, the phage line remains flat at 14. However, the incubation also grows the bacteria. After about forty minutes, the number of bacteria begins to increase as shown at 22 and accelerates in region 24. The point at which bacteriophage begin to rapidly find and infect the host bacteria occurs at a quite narrow bacterial concentration range 28 owing to diffusion and binding effects. In the example of FIG. 1a, this occurs at a bacterial concentration of about 10⁵ to 10⁶ bacteria per ml. The number of bacteriophage does not increase immediately, because it takes some time for the bacteriophage to multiply after infecting the bacteria. The bacteriophage rise becomes exponential at about 240 minutes, which causes the bacterial growth to decelerate in the region 25 and then turn around at 26. After the bacteria concentration peaks, the phage curve flattens to create a knee 18 at about 330 minutes and peaks at about 360 minutes. The number of bacteria steeply decreases in the region 27 as the phage infect and kill the bacteria and the phage number continues to increase. By 360 minutes, the phage versus time curve is essentially flat since all but a minor portion of the bacteria are dead.

FIG. 1b shows a similar characteristic for bacterial markers. The figure includes a graph 31 showing the number of bacteria per minute being lysed by the phage in FIG. 1a. As bacteria are lysed, the number of bacterial markers increases proportionally to the total number of bacteria that have been lysed by the phage as shown in graph 32.

The inventors have determined that the graphs 10 and 32 are not just qualitative. That is, the time it takes for the quantity of bacteriophage or bacterial marker to reach a specific level T_{P} depends primarily on the initial concentration of the target microorganism in the sample. The measured time T_{P} can be chosen to correspond to a distinct marker concentration. It can be the time it takes for the bacteriophage concentration to begin flattening off at the knee 18 or when its concentration peaks at 15. In FIG. 1a, the time T_{P} corresponds to the time when the phage concentration goes beyond a threshold level 30 and is about 300 minutes. Preferably, the threshold level 30 corresponds to a time at which the bacteriophage concentration is increasing rapidly as shown in FIG. 1a. The threshold level 30 must exceed the initial concentration of bacteriophage added to the sample. In a preferred embodiment, the threshold level 30 corresponds to a value that equals or exceeds the detection limit of the detector used to detect bacteriophage in the sample and the initial bacteriophage concentration is kept below that detection limit. If bacterial markers are measured, the time T_{P} might correspond to a time when the bacterial marker concentration goes beyond a threshold level 35 as shown in FIG. 1b. Preferably, the threshold level 35 corresponds to a time at which the bacterial marker concentration is increasing rapidly.

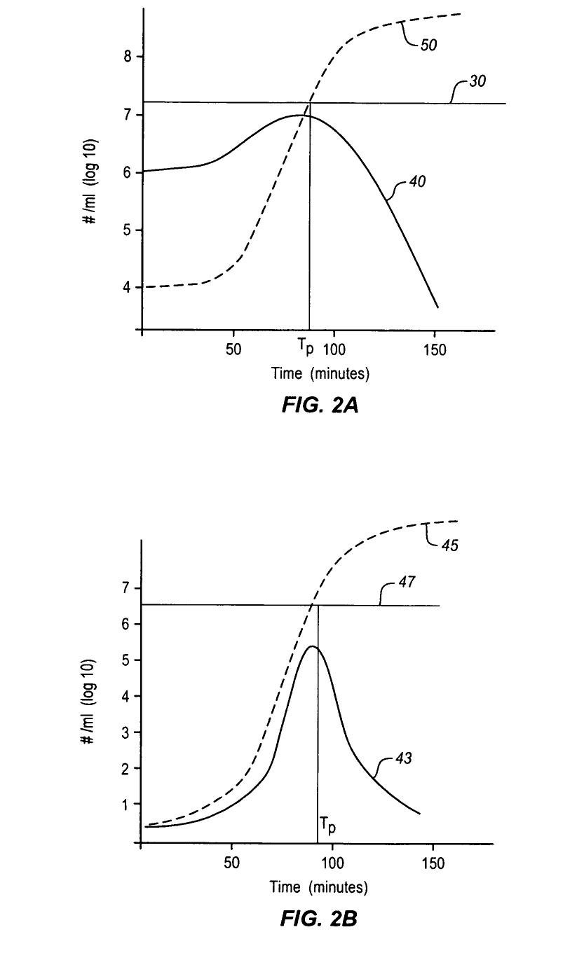

The time T_{P} it takes for the bacteriophage versus time curve to reach the chosen threshold level depends on the concentration of bacteria at time zero, the lag time before normal bacterial growth occurs, the doubling time of the specific microorganism, the number of bacteriophage added, and the incubation conditions. For a particular microorganism and microorganism-specific bacteriophage, a fixed initial bacteriophage concentration, and for identical incubation conditions, the time T_{P} will depend only on the initial concentration of target microorganisms present in the sample, the lag time before normal growth occurs, and the doubling time of the microorganism. For a given type of sample matrix, lag times for a microorganism vary only moderately. Doubling times vary somewhat for different strains of a given bacteria, but this variation is not usually large. Thus, by adding a predetermined number of bacteriophage at time zero, the concentration of the target microorganism present in a sample can be estimated by measuring T_{P}. For example, FIG. 2a shows the results for a sample that is identical to the sample of FIG. 1, except that the bacteria concentration at the start was 10⁶ bacteria per ml. The bacteria concentration is shown in curve 40, while the bacteriophage concentration is shown in curve 50. In this case, T_{P} is selected to be the time to reach the bacteriophage threshold 30 and is about 90 minutes. Similarly, FIG. 2b shows a graph 43 of the number of bacteria being lysed per minute by phage and a graph 45 of the concentration of a bacterial marker 45 over time for the same sample.

The prior art has not recognized the above fact because the prior art generally describes the usage of high concentrations of bacteriophage (>10⁸). In this case, the time T_{P} will depend only weakly, if at all, on microorganism concentration and will depend more strongly on the type of bacteriophage and microorganism.

The inventors have found that the process of the invention works best when the number of bacteriophage added to the sample is kept low, that is, at 10⁷ bacteriophage per ml or less, and more preferably, at 10⁶ bacteriophage per ml or less. Most preferably, the number of phage are below the level that can be detected using the phage marker, which depends on the detection method, but maybe as low as 5×10⁵ bacteriophage per milliliter or lower. If the concentration of phage and bacteria are small, the probability of a phage and a bacterium colliding and initiating the phage amplification process is low. The inventors have found that, even though this is a fundamentally random process, it is predictable. No matter how low the number of phage, eventually a peak will occur if there are target bacteria in the sample. The primary variable is how long it will take to appear.

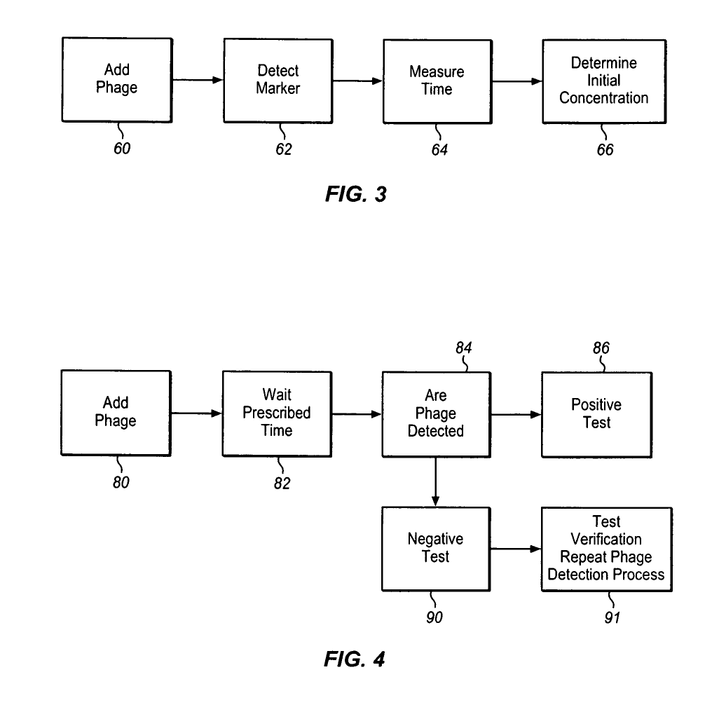

FIG. 3 illustrates a preferred embodiment of the process according to the invention. At 60, a predetermined concentration of bacteriophage specific to a target microorganism is added to a sample for which it is desired to know the concentration of the target microorganism. At 62, the bacteriophage or bacterial marker is detected at threshold level 30 or 35 (FIGS. 1(a) and 1(b)), respectively. The time to reach the threshold level 30 or 35 is measured at 64. This time then is used to determine the initial concentration of microorganisms in the sample at 66. Preferably, prior to the test, a table of time to the detection point versus microorganism concentration is made based on a range of measured results. If a time is between points on the table, then extrapolation may be used to determine the initial concentration.

FIG. 4 is a flow chart illustrating another preferred embodiment of the invention. This embodiment is particularly useful in determining if a minimum level of microorganisms is present in the sample. At 80, a predetermined concentration of bacteriophage specific to a target microorganism is added to the sample. The sample then is allowed to incubate at 82 for a specified time period, after which it is known from curves such as 10 and 50 or 32 and 45 that the bacteriophage or bacterial marker will be detectable if the concentration of the target microorganism is above the threshold. It then is determined if the marker is detectable at 84. If the marker is detected, the test is declared positive at 86, and the initial concentration of the target microorganism was at the minimum level or above it. If the marker is not detected, the test is declared negative at 90, and the initial concentration of the target microorganism is determined to be less than the minimum level. As a test verification, at 91, the bacteriophage or bacterial detection process is repeated at a later time. As an example of the foregoing embodiment, many people are carriers for Strep pneumoniae bacteria. If the concentration of bacteria in a person's upper respiratory tract is less than 10³ bacteria per ml or perhaps 10⁴ bacteria per ml, there is no immediate health problem. However, if the concentration of bacteria exceeds 10⁵ or 10⁶ bacteria per ml, they will likely be experiencing health problems for which medical care is advisable. Thus, if a threshold time T_{T} is selected such that an initial concentration of Strep pneumoniae bacteria of 3×10⁴ will enable a detectable level of S. pneumoniae specific bacteriophage or S. pneumoniae marker to be detectable at time T_{T}, and no such marker is detected at time T_{T}, then there is no immediate health problem. If the person for whom the test is performed is known to be a carrier, and at later time T_{L} at which it is known that markers should be detected for this person, but no bacteriophage or bacterial markers are detected, then the test will be determined to be defective and the test can be repeated. If bacteriophage or bacterial markers are detected at this time T_{L}, then the test is verified.

The methods of the invention may also be used in an antibiotic susceptibility test. However, it is preferred that bacteriophage markers are used in the assay rather than bacterial markers because many antibiotics lyse bacteria just as bacteriophage do and thereby release the same bacterial markers. The release of the antibiotic-induced bacterial markers could disturb the assay results.

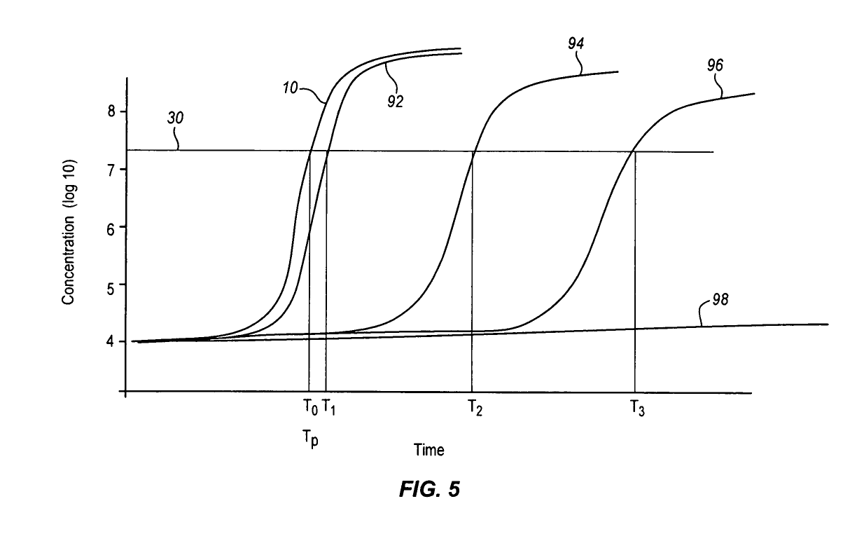

The basis for the antibiotic susceptibility test is illustrated in FIG. 5. If an antibiotic is added to a sample to which a target specific phage is also added, and the target microorganism is present, then the antibiotic will delay phage replication by an amount that correlates with the effectiveness of the antibiotic against the microorganism. The phage concentration curve versus time will indicate the efficacy of the specific antibiotic. That is, to the degree that the antibiotic slows the growth of the bacteria or kills it, the phage will have fewer bacteria to infect at a given time after the assay starts, and the phage concentration increase will take a longer time to develop. As discussed in more detail below, this is particularly true for antibiotics that disturb nucleic acid (e.g., DNA or RNA) replication or protein synthesis of the bacteria, since phage reproduction relies on these bacterial processes to proceed. FIG. 5 shows the phage concentration curve 10 of FIG. 1 as modified by four different concentrations of a given antibiotic: A, B, C, and D. In each curve, the time at which the phage concentration exceeds the threshold level 30 is inversely correlated to the effectiveness of the antibiotic. In FIG. 5, antibiotic concentration A associated with the curve 92 essentially is ineffective against the microorganism, since the phage concentration versus time curve is hardly altered, and the time T₁ is very similar to the time T₀ corresponding to the no-antibiotic curve 10. Antibiotic concentration B associated with the curve 94 is higher than concentration A and is more effective, since the peak has been delayed until a time T₂ that is significantly later than the time T₀. Antibiotic concentration C associated with the curve 96 is higher still and is even more effective against the bacteria, since the phage threshold level 30 is detectable only at a much later time. Finally, an even higher antibiotic concentration D associated with the curve 98 is very effective against the bacteria, since the threshold level 30 is never reached. A similar test can be carried out for different antibiotics.

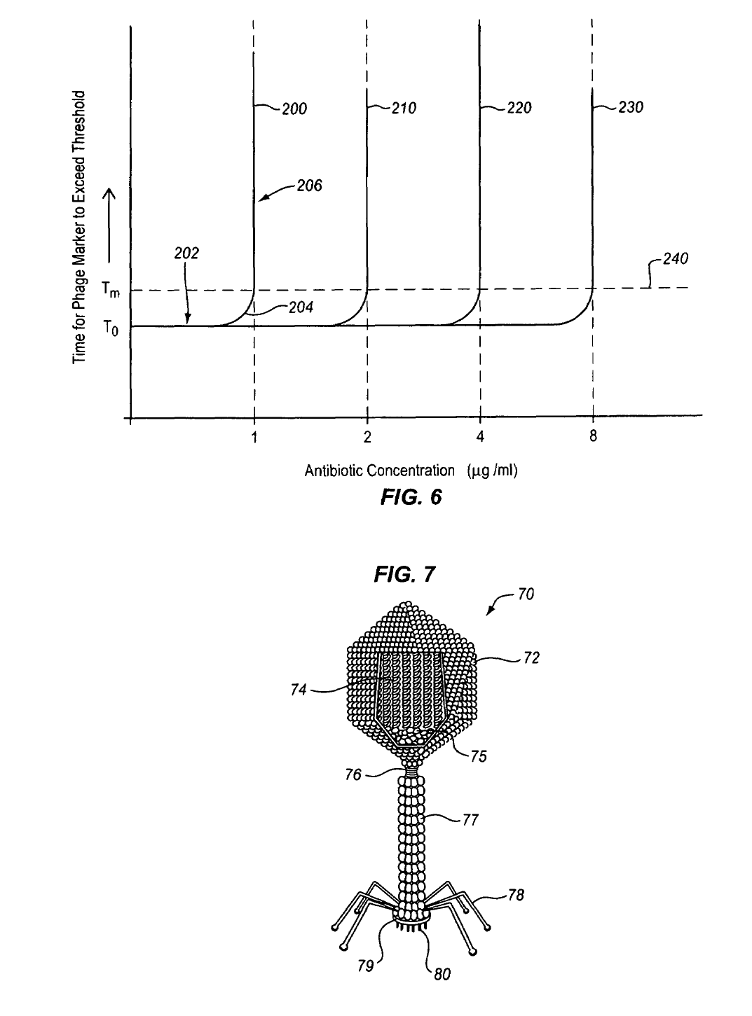

FIG. 6 illustrates the relationship between the times at which a bacteriophage marker exceeds a threshold level as a function of antibiotic concentration in a sample. Curve 200 shows the relationship for a specific bacterial strain A. At an antibiotic concentration near zero, the measured time T is a constant value of T₀. As the antibiotic concentration is increased, the measured time begins to increase at 204. As the antibiotic concentration approaches a critical value, the measured time begins to increase rapidly at 206. Beyond the critical antibiotic concentration, the bacteriophage marker never exceeds the threshold level. The critical antibiotic concentration at which phage replication is inhibited is related to the Minimum Inhibitory Concentration (MIC) of the bacterial strain. For curve 200 in FIG. 6, the strain's MIC is 2; in other words, the phage marker is amplified at a concentration of 1 ug/ml but does not amplify at the next antibiotic concentration level of 2. For strains with higher MIC values, a very similar curve is obtained with higher critical antibiotic concentrations. The curve 210 corresponds to a strain having an MIC of 4. Similarly, curves 220 and 230 correspond to strains with MICs of 8 and 16, respectively.

A simple test of the susceptibility or resistance of a given bacteria to an antibiotic can be designed using the curves shown in FIG. 6. A fixed concentration of antibiotic such as 2 ug/ml is added to a sample such that the antibiotic may inhibit normal bacterial growth or even kill the bacteria. A fixed concentration of a phage specific to the target bacteria is added to the sample. Preferably, the phage concentration is below the detection limit. At a fixed time Tₘ as shown in FIG. 6, the phage concentration is measured using the methods described herein. If the phage concentration has increased from the initial concentration at the measurement time Tₘ, it indicates that the tested antibiotic in the tested concentration did not adequately inhibit bacterial growth and phage replication. Therefore, the test would indicate that the bacteria are resistant to the antibiotic at the concentration used; i.e., the MIC for that antibiotic is greater than the tested antibiotic concentration. By selecting appropriate starting antibiotic concentrations, this method can be used to determine if a bacteria is resistant to a given concentration (bacteriophage marker detected at or above the threshold level at the time Tₘ) or susceptible (bacteriophage marker NOT detected at or above the threshold level at time Tₘ).

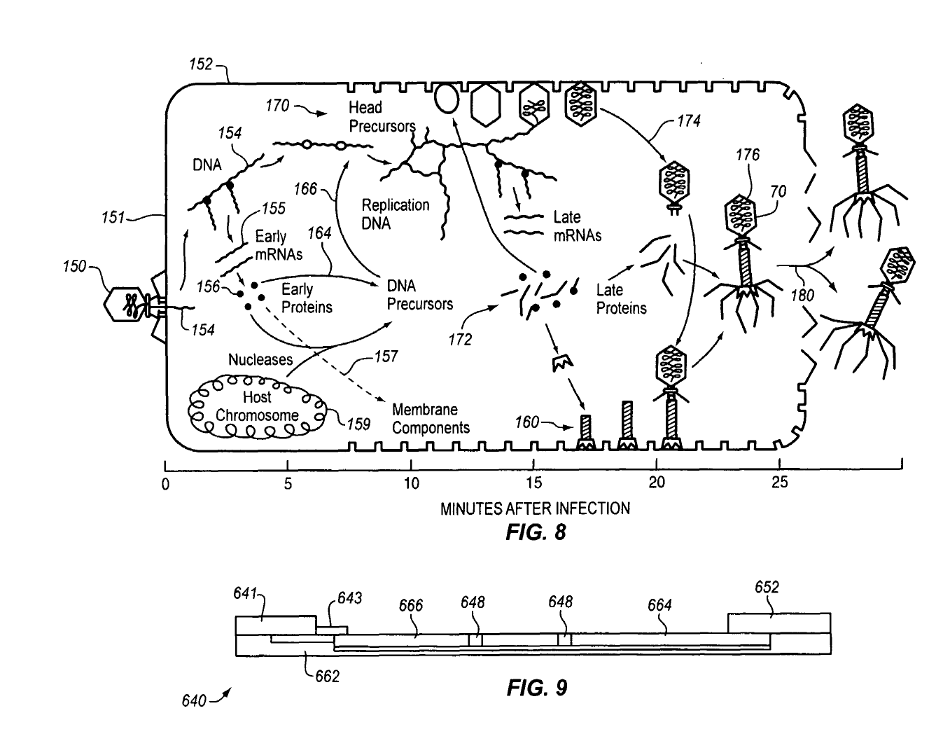

As indicated above, the antibiotic susceptibility or resistance test works particularly well for antibiotics that inhibit the DNA, RNA, or protein production. This is illustrated in connection with FIGS. 7 and 8. FIG. 7 illustrates a typical phage 70, and FIG. 8 illustrates a typical phage reproduction process as a function of time. Structurally, a bacteriophage 70 comprises a protein shell or capsid 72, sometimes referred to as a head, which encapsulates the viral nucleic acids 74, i.e., the DNA and/or RNA. A bacteriophage may also include internal proteins 75, a neck 76, a tail sheath 77, tail fibers 78, an end plate 79, and pins 80. The capsid 72 is constructed from repeating copies of one or more proteins. Referring to FIG. 8, when a phage 150 infects a bacterium 152, it attaches itself to a particular site on the bacterial wall or membrane 151 and injects its nucleic acid 154 into that bacterium, inducing it to replicate tens to thousands of phage copies. The DNA evolves to early mRNAS 155 and early proteins 156, some of which become membrane components along line 157 and others of which utilize bacteria nucleases from host chromosomes 159 to become DNA precursors along line 164. Others migrate along the direction 170 to become head precursors that incorporate the DNA along line 166. The membrane components evolve along the path 160 to form the sheath, end plate, and pins. Other proteins evolve along path 172 to form the tail fibers. When formed, the head releases from the membrane 151 and joins the tail sheath along path 174, and then the tail sheath and head join the tail fibers at 176 to form the bacteriophage 70. Some bacteriophage, called lytic bacteriophage, rupture the host bacterium, shown at 180, releasing the progeny phage into the environment to seek out other bacteria.

From the above, it is evident that, if the antibiotic inhibits DNA (or RNA) replication within the bacteria, then the bacteriophage reproduction will also be directly inhibited because the phage will not be able to make the copies of its DNA or RNA from which, when expressed, the many parts of the phage are built. Antibiotic classes that inhibit DNA replication include: flouroquinilones, such as levofloxacin and ciprofloxacin, and rifampin. Similarly, if the antibiotic inhibits bacterial protein synthesis, then it will also directly inhibit phage replication because the phage will not be able to generate the many proteins needed to build new phage particles including capsid proteins. Antibiotic classes that block protein synthesis include: macrolides, aminoglycosides, tetracyclines, streptogramins, everninomycins, oxazolidinones, and lincosamides.

The methods described herein can be used with antibiotics that do not inhibit DNA (or RNA) replication or protein synthesis. Such antibiotics include those that inhibit cell wall biosynthesis such as penicillins, cephalosporins, carbapenems, and glycopeptides. While these antibiotics do not directly inhibit phage replication, they do inhibit it indirectly by disturbing various bacterial metabolic activities such that the bacteria themselves grow more slowly, not at all, or they die. A table describing some antibiotics classes and listing particular antibiotics in each class is shown in Appendix 1. All antibiotics when used at an effective concentration either inhibit cell growth or kill bacteria. These are called bacteriostatic and bactericidal antibiotics respectively. The methods described herein can be used with either type of antibiotic; however, the methods are easier to apply to bactericidal antibiotics because phage cannot replicate in dead bacteria.

The methods described for determining the antibiotic resistance or susceptibility of a given bacteria may require that the initial concentration of bacteria in the sample is either known or is measured. If it is not, then the measured time to detect phage concentrations that exceed a specific threshold level cannot be ascribed to the antibiotic alone. For example, the measured time will be longer if the starting sample has 10 bacteria per ml versus 10⁵ bacteria per ml. A simple way of measuring the initial bacterial concentration using the methods described herein and illustrated in FIG. 3 and 4 is to run a duplicate sample with no antibiotic. The measured time T₀ will be the baseline value shown in FIG. 5. Any increase in the measured time for the sample containing the antibiotic is due solely to the antibiotic. Care must also be taken with the initial bacterial concentration in the sample. If it is higher than the level at which phage replication can occur quickly as described in reference to FIG. 1, then phage replication may occur despite the presence of the antibiotic because the antibiotic doesn't kill the bacteria quickly enough. This may be the case with many clinical samples that typically contain high bacterial loads such as positive blood culture sample and samples associated with urinary or respiratory tract infections. For antibiotics that directly inhibit phage replication, this may not be a concern—phage replication cannot occur no matter the initial bacterial concentration. For those that do not, then either 1) the sample must be diluted such that bacterial concentrations are reduced to level at which phage replication will not occur immediately, or 2) the antibiotic must be added to the sample in advance of the phage so that the antibiotic has time to kill some portion of the susceptible bacteria.

Generally, many antibiotic susceptibility tests can be carried out simultaneously, with each different antibiotic and/or different antibiotic concentration being added to a different and separate sample, with all samples being identical except for the antibiotic. Further details of antibiotic susceptibility studies maybe found in United States Patent Application 2005/0003346 A1 published Jan. 6, 2005 on an invention of Voorhees et al., which patent publication is incorporated by reference herein to the same extent as though fully disclosed herein.

Any detection method or apparatus that detects bacteriophage or bacterial markers when a specific microorganism is present can be used in the invention, that is, to detect the markers in processes 62, 84, and 91 and in the antibiotic susceptibility tests described above. Preferred methods are immunoassay methods utilizing antibody-binding events to produce detectable signals including ELISA, radioimmunoassay, immunoflouresence, lateral flow immunochromatography (LFI, flow-through assay, and the use of a SILAS surface which changes color as a detection indicator. Other methods are nucleic acid amplification-based assays, DNA probe assays, aptamer-based assays, mass spectrometry, such as matrix-assisted laser desorption/ionization with time-of-flight mass spectrometry (MALDI-TOF-MS), referred to herein as MALDI, flow, and cytometry. One immunoassay method, LFI, is discussed in detail below in connection with FIG. 8.

A cross-sectional view of the lateral flow strip 640 is shown in FIG. 9. The lateral flow strip 640 preferably includes a sample application pad 641, a conjugate pad 643, a substrate 664 in which a detection line 646 and an internal control line 648 are formed, and an absorbent pad 652, all mounted on a backing 662, which preferably is plastic. The substrate 664 preferably is a porous mesh or membrane. It is made by forming lines 643, 646, and optionally line 648, on a long sheet of said substrate, then cutting the substrate in a direction perpendicular to the lines to form a plurality of substrates 664. The conjugate pad 643 contains beads, each of which has been conjugated to a first antibody forming first antibody-bead conjugates. The first antibody selectively binds to the marker in the test sample. Detection line 646 and control line 648 are both reagent lines, and each form an immobilization zone; that is, they contain a material that interacts in an appropriate way with the marker. In the preferred embodiment, the interaction is one that immobilizes the marker. Detection line 646 preferably comprises immobilized secondary antibodies, with antibody line 646 perpendicular to the direction of flow along the strip, and being dense enough to capture a significant portion of the marker in the flow. The second antibody also binds specifically to the marker. The first antibody and the second antibody may or may not be identical. Either may be polyclonal or monoclonal antibodies. Optionally, strip 640 may include a second reagent line 648 including a third antibody. The third antibody may or may not be identical to one or more of the first and second antibodies. Second reagent line 648 may serve as an internal control zone to test if the assay functioned properly.

One or more drops of a test sample are added to the sample pad. The test sample preferably contains parent phage as well as progeny phage and bacterial markers if the target bacterium was present in the original raw sample. The test sample flows along the lateral flow strip 640 toward the absorbent pad 652 at the opposite end of the strip. As the bacteriophage or bacterial markers flow along the conjugate pad toward the membrane, they pick up one or more of the first antibody-bead conjugates forming phage-bead complexes. As the phage-bead complexes move over row 646 of second antibodies, they form an immobilized and concentrated first antibody-bead-marker-second antibody complex. If enough marker-bead complexes bind to the row 646 of immobilized second antibodies, a line becomes detectable. The detectability of the line depends on the type of bead complex. As known in the art, antibodies may be conjugated with a colored latex bead, colloidal gold particles, or a fluorescent magnetic, paramagnetic, superparamagnetic, or supermagnetic marker, as well as other markers, and maybe detected either visually or otherwise as a color, or by other suitable indicator. A line indicates that the target microorganisms were present in the raw sample. If no line is formed, then the target microorganisms were not present in the raw sample or were present in concentrations too low to be detected with the lateral flow strip 640. For this test to work reliably, the concentration of parent phage added to the raw sample should be low enough such that the parent phage alone are not numerous enough to produce a visible line on the lateral flow strip if it is designed to detect bacteriophage markers. The antibody-bead conjugates are color moderators that are designed to interact with the bacteriophage or bacterial markers. When they are immobilized in the immobilization zone 646, they cause the immobilization zone to change color. A more complete description of the lateral flow strip and process are given in United States Patent Application Publication No. 2005/0003346 published Jan. 6, 2005, which is incorporated herein by reference to the same extent as though fully disclosed herein.

Many other phage-based methods and apparatus maybe used to identify the microorganism and/or to determine the antibiotic susceptibility, i.e., used or partially used in processes 62, 84, 91 etc. Examples of such processes are disclosed in the following publications:

United States Patents:
U.S. Pat. No. 4,104,126 issued Aug. 1, 1978 to David M. Young
U.S. Pat. No. 4,797,363 issued Jan. 10, 1989 to Teodorescu et al.
U.S. Pat. No. 4,861,709 issued Aug. 29, 1989 to Ulitzur et al.
U.S. Pat. No. 5,085,982 issued Feb. 4, 1992 to Douglas H. Keith
U.S. Pat. No. 5,168,037 issued Dec. 1, 1992 to Entis et al.
U.S. Pat. No. 5,498,525 issued Mar. 12, 1996 to Rees et al.
U.S. Pat. No. 5,656,424 issued Aug. 12, 1997 to Jurgensen et al.
U.S. Pat. No. 5,679,510 issued Oct. 21, 1997 to Ray et al.
U.S. Pat. No. 5,723,330 issued Mar. 3, 1998 to Rees et al.
U.S. Pat. No. 5,824,468 issued Oct. 20, 1998 to Scherer et al.
U.S. Pat. No. 5,888,725 issued Mar. 30, 1999 to Michael F. Sanders
U.S. Pat. No. 5,914,240 issued Jun. 22, 1999 to Michael F. Sanders
U.S. Pat. No. 5,958,675 issued Sep. 28, 1999 to Wicks et al.
U.S. Pat. No. 5,985,596 issued Nov. 16, 1999 to Stuart Mark Wilson
U.S. Pat. No. 6,090,541 issued Jul. 18, 2000 to Wicks et al.
U.S. Pat. No. 6,265,169 B1 issued Jul. 24, 2001 to Cortese et al.
U.S. Pat. No. 6,300,061 B1 issued Oct. 9, 2001 to Jacobs, Jr. et al.
U.S. Pat. No. 6,355,445 B2 issued Mar. 12, 2002 to Cherwonogrodzky et al.
U.S. Pat. No. 6,428,976 B1 issued Aug. 6, 2002 to Chang et al.
U.S. Pat. No. 6,436,652 B1 issued Aug. 20, 2002 to Cherwonogrodzky et al.
U.S. Pat. No. 6,436,661 B1 issued Aug. 20, 2002 to Adams et al.
U.S. Pat. No. 6,461,833 B1 issued Oct. 8, 2002 to Stuart Mark Wilson
U.S. Pat. No. 6,524,809 B1 issued Feb. 25, 2003 to Stuart Mark Wilson
U.S. Pat. No. 6,544,729 B2 issued Apr. 8, 2003 to Sayler et al.
U.S. Pat. No. 6,555,312 B1 issued Apr. 29, 2003 to Hiroshi Nakayama

United States Published Applications:
2002/0127547 A1 published Sep. 12, 2002 by Stefan Miller
2004/0121403 A1 published Jun. 24, 2004 by Stefan Miller
2004/0137430 A1 published Jul. 15, 2004 by Anderson et al.
2005/0003346 A1 published Jan. 6, 2005 by Voorhees et al.

Foreign Patent Publications:
EPO 0 439 354 A3 published Jul. 31, 1991 by Bittner et al.
WO 94/06931 published Mar. 31, 1994 by Michael Frederick Sanders
EPO 1 300 082 A2 published Apr. 9, 2003 by Michael John Gasson
WO 03/087772 A2 published Oct. 23, 2003 by Madonna et al.

Other Publications:
Favrin et al., “Development and Optimization of a Novel Immunomagnetic Separation-Bacteriophage Assay for Detection of Salmonella enterica Serovar Enteritidis in Broth”, Applied and Environmental Microbiology, January 2001, pp. 217-224, Volume 67, No. 1.
   All of the forgoing publications are hereby incorporated by reference to the same extent as though fully disclosed herein. Any other bacteriophage-based process may be used as well.

A feature of the invention is that the bacteriophage-based method taught herein distinguishes between live and dead bacteria. This is essential for antibiotic susceptibility tests, food applications where the food has been irradiated, or any other application where dead bacteria may be present. Thus, the invention provides significant advantages over other methods, such as nucleic acid-based technologies (PCR, etc.) or immunological tests that look for bacterial components rather than phage components because the former cannot readily distinguish between live and dead bacteria.

Another feature of the invention is that the bacteriophage-based method is simpler and less expensive than other tests. This permits a detection system that remains relatively inexpensive, while at the same time being significantly faster. A further feature of the invention is that the antibiotic susceptibility subprocess. is also simple and can follow protocols that are similar to conventional antibiotic susceptibility processes; thus, little training is required to update to the bacteriophage-based susceptibility tests, both of which contribute to keeping the cost low.

There has been described a microorganism quantification method which is specific to a selected organism, which is sensitive, simple, fast, and/or economical, and having numerous novel features. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described, without departing from the inventive concepts. For example, in the process of the invention, many samples, each with a different predetermined amount of parent bacteriophage, could be used. Then the first one to show a detectable bacteriophage marker level would also indicate the initial quantity of the target microorganism; or, after a certain time, several of the results could be used to provide a more accurate determination of the initial quantity of the target microorganism. Equivalent structures and processes may be substituted for the various structures and processes described; the subprocesses of the inventive method may, in some instances, be performed in a different order, or a variety of different materials and elements may be used. Consequently, the invention is to be construed as embracing each and every novel feature and novel combination of features present in and/or possessed by the microorganism detection apparatus and methods described.

What is claimed as new and desired to be protected by Letters Patent of the United States is:

1. A method of determining if a threshold concentration of a target microorganism is present in a sample to be tested, said method comprising:
(a) combining with said sample a predetermined amount of parent bacteriophage capable of infecting said target microorganism to create a bacteriophage-exposed sample;
(b) providing incubation conditions to said bacteriophage-exposed sample sufficient to allow said parent bacteriophage to infect said target microorganism,;
(c) waiting a predetermined time period such that, if said target microorganism is present in said sample at or above a threshold concentration, a marker will be amplified in said sample; and
(d) assaying said exposed sample to determine the level of said marker.

2. A method as in claim 1 wherein said target microorganism is a bacterium.

3. A method as in claim 1 wherein said parent bacteriophage has been genetically modified to add said marker.

4. A method as in claim 1 wherein said marker is a bacteriophage marker.

5. A method as in claim 4 wherein said bacteriophage marker comprises an element selected from the group consisting of said bacteriophage, bacteriophage nucleic acid, bacteriophage protein, and a portion of a bacteriophage nucleic acid or a bacteriophage protein.

6. A method as in claim 4 wherein said parent bacteriophage is combined in an amount below the detection limit of said bacteriophage marker.

7. A method as in claim 1 wherein said marker is a bacterial marker and comprises an element selected from the group consisting of: cell wall debris, bacterial nucleic acids, proteins, or enzymes that are released when a phage lyses the bacteria.

8. A method as in claim 1 wherein said assaying comprises a colorimetric test.

9. A method as in claim 1 wherein said assaying comprises one or more tests selected from the group consisting of immunoassay methods, nucleic acid amplification-based assays, DNA probe assays, aptamer-based assays, mass spectrometry, including MALDI, and flow cytometry.

10. A method as in claim 9 wherein said immunoassay methods are selected from the group consisting of ELISA, radioimmunoassay, immunoflouresence, lateral flow immunochromatography (LFI), flow-through assay, and a test using a SILAS surface.

11. A method of determining the initial quantity of a microorganism present in a sample, said method comprising:
(a) combining with said sample a predetermined amount of parent bacteriophage capable of infecting said target microorganism to create a bacteriophage-exposed sample;
(b) providing incubation conditions to said bacteriophage-exposed sample sufficient to allow said parent bacteriophage to infect said target microorganism and create an amplified marker in said bacteriophage-exposed sample;
(c) assaying said marker in said exposed sample to determine a marker level in said sample;
(d) measuring a reaction time associated with said marker level; and
(e) determining said initial quantity of said microorganism present in said sample using said marker level and said measured reaction time.

12. A method as in claim 11 wherein said initial quantity comprises the concentration of said microorganism in said sample at the time of adding said parent bacteriophage.

13. A method as in claim 11 wherein said target microorganism is a bacterium

14. A method as in claim 11 wherein said parent bacteriophage has been genetically modified to add said marker.

15. A method as in claim 11 wherein said determining comprises:
providing a table correlating said reaction time to said initial quantity; and selecting said initial quantity from said table.

16. A method as in claim 15 wherein said table also correlates said predetermined amount of parent bacteriophage to said initial quantity.

17. A method as in claim 11 wherein:
said measuring comprises waiting a predetermined time;
said assaying comprises establishing if said sample contains a detectable amount of said marker, and
said determining comprises ascertaining that said initial quantity is below a threshold value.

18. A method as in claim 11 wherein said marker is a bacteriophage marker.

19. A method as in claim 18 wherein said bacteriophage marker comprises an element selected from the group consisting of said bacteriophage, bacteriophage nucleic acid, bacteriophage protein, and a portion of a bacteriophage nucleic acid or a bacteriophage protein.

20. A method as in claim 17 wherein said parent bacteriophage is added in an amount below the detection limit of said bacteriophage marker, and said marker level is at or near said detection limit.

21. A method as in claim 11 wherein said marker is a bacterial marker and comprises an element selected from the group consisting of: cell wall debris, bacterial nucleic acids, proteins, or enzymes that are released when a phage lyses the bacteria.

22. A method as in claim 11 wherein said assaying comprises a colorimetric test.

23. A method as in claim 11 wherein said assaying comprises one or more tests selected from the group consisting of immunoassay methods, nucleic acid amplification-based assays, DNA probe assays, aptamer-based assays, mass spectrometry, including MALDI, and flow cytometry.

24. A method as in claim 23 wherein said immunoassay methods are selected from the group consisting of ELISA, radioimmunoassay, immunoflouresence, lateral flow immunochromatography (LFI), flow-through assay, and a test using a SILAS surface.

25. A method of determining the susceptibility or resistance of a target microorganism to an antibiotic, said method comprising:
(a) combining with said target microorganism and said antibiotic a predetermined amount of parent bacteriophage capable of infecting said target microorganism to create a bacteriophage-exposed sample;
(b) providing incubation conditions to said bacteriophage-exposed sample sufficient to allow said parent bacteriophage to infect said target microorganism;
(c) waiting a predetermined time period such that, if said target microorganism is not susceptible to said antibiotic, a bacteriophage marker will be amplified in said sample; and
(d) assaying said exposed sample to determine the level of said bacteriophage marker as an indication of the susceptibility of said microorganism to said antibiotic.

26. A method as in claim 25 wherein said parent bacteriophage is combined in an amount below the detection limit of said bacteriophage marker.

27. A method as in claim 25 wherein said antibiotic inhibits nucleic acid replication.

28. A method as in claim 27 wherein said antibiotic is selected from the group consisting of: flouroquinilones, such as levofloxacin and ciprofloxacin, and rifampin.

29. A method as in claim 25 wherein said antibiotic inhibits protein synthesis.

30. A method as in claim 29 wherein said antibiotic is selected from the group consisting of: macrolides, aminoglycosides, tetracyclines, streptogramins, everninomycins, oxazolidinones, and lincosamides.

31. A method as in claim 25 wherein said assaying comprises a colorimetric test.

32. A method as in claim 25 wherein said assaying comprises one or more tests selected from the group consisting of immunoassay methods, nucleic acid amplification-based assays, DNA probe assays, aptamer-based assays, mass spectrometry, including MALDI, and flow cytometry.

33. A method as in claim 32 wherein said immunoassay methods are selected from the group consisting of ELISA, radioimmunoassay, immunoflouresence, lateral flow immunochromatography (LFI), flow-through assay, and a test using a SILAS surface.

34. A method as in claim 25 wherein said combining comprises diluting the concentration of said target microorganism to a level at which said bacteriophage infection will not occur immediately.

35. A method of determining the susceptibility or resistance of a target microorganism to an antibiotic, said method comprising:
(a) combining said target microorganism, said antibiotic, and a predetermined amount of parent bacteriophage capable of infecting said target microorganism to create a bacteriophage-exposed sample;
(b) providing incubation conditions to said bacteriophage-exposed sample sufficient to allow said parent bacteriophage to infect said target microorganism and create an amplified bacteriophage marker in said bacteriophage-exposed sample;
(c) assaying said bacteriophage marker in said exposed sample to determine a marker level in said sample;
(d) measuring a reaction time associated with said marker level; and
(e) determining the susceptibility of said target microorganism to said antibiotic using said marker level and said measured reaction time.

36. A method as in claim 35 wherein said antibiotic inhibits nucleic acid synthesis.

37. A method as in claim 36 wherein said antibiotic is selected from the group consisting of: flouroquinilones, such as levofloxacin and ciprofloxacin, and rifampin.

38. A method as in claim 35 wherein said antibiotic inhibits protein synthesis.

39. A method as in claim 38 wherein said antibiotic is selected from the group consisting of: macrolides, aminoglycosides, tetracyclines, streptogramins, everninomycins, oxazolidinones, and lincosamides.
